(19) United States
(12) Patent Application Publication
(10) Pub. No.: US 2007/0093807 A1
(43) Pub. Date: Apr. 26, 2007

(54) FINGER TIP ELECTROSURGICAL MEDICAL DEVICE

(76) Inventors: Chester O. Baxter III, Loveland, OH (US); Craig N. Faller, Milford, OH (US); Kevin L. Houser, Springboro, OH (US); Jorge Gutierrez, Cincinnati, OH (US); William T. Donofrio, Cincinnati, OH (US); Jerome R. Morgan, Cincinnati, OH (US); Jeffrey J. Vaitekunas, St. Paul, MN (US); Gregory W. Johnson, Milford, OH (US); Vance VanDrake III, Cincinnatti, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/614,203

(22) Filed: Dec. 21, 2006

Related U.S. Application Data
(63) Continuation of application No. 10/659,418, filed on Sep. 10, 2003, now abandoned.
(60) Provisional application No. 60/412,072, filed on Sep. 19, 2002.
(60) Provisional application No. 60/425,401, filed on Nov. 12, 2002.

(51) Int. Cl. A61B 18/14

(52) U.S. Cl. 606/41; 606/45; 606/49

(57) ABSTRACT

Described is a fingertip electrosurgical medical device useful for tissue grasping and tissue cutting, coagulating, welding and ablating in open and laparoscopic surgery applications. The electrosurgical medical device has a finger cuff assembly that attaches to the distal end of a surgeon's finger. An electrode is attached to, or is intrinsically part of, the finger cuff assembly. This electrode is connected to at least one electrically conductive wire that is adapted to transmit electric current to the electrode. The electrode may take any number of shapes depending upon the surgical procedure. Generally, two finger cuff assemblies are used in an opposable relationship to facilitate energy transfer or tissue grasping functions performed by the surgeon.

### CROSS REFERENCE TO RELATED APPLICATIONS

The present application is a continuation of U.S. patent application Ser. No. 10/659,418, filed on Sep. 10, 2003, which claims the priority benefit of U.S. provisional patent application Ser. Nos. 60/412,072, filed on Sep. 19, 2002 and 60/425,401, filed on Nov. 12, 2002, both of which are incorporated herein by reference.

### FIELD OF THE INVENTION

The present invention relates, in general, to medical devices and, more particularly, to fingertip electrosurgical medical devices for use in grasping, cutting, coagulating, tissue welding and tissue ablation medical procedures.

### BACKGROUND OF THE INVENTION

Electrosurgery is one form of a surgical cutting and coagulating procedure.

Electrosurgery has two primary modes monopolar and bipolar. Monopolar surgery uses an instrument with a single electrode such as a source, and a grounding pad (return electrode) as the means to complete the electrical circuit of a surgical generator to the patient. In contrast, bipolar instruments include two electrodes in close proximity to each other. Typically, one electrode is a supply electrode and the other electrode is a return electrode.

Bipolar electrosurgical procedures are typically done with a scissors or grasper device that require a surgeon to use their depth perception to grasp hold of tissue and vessels. These electrosurgical instruments have end-effectors that are often limited to up and down or side-to-side actuation due to mechanical design constraints. This limited range of motion may be undesirable to a surgeon working in an area that requires a complex series of movements. In situations such as this, it is desirable to have electrosurgical instruments that have a wide range of motion to allow for complex surgical articulation.

Further, the mechanical nature of many electrosurgical instruments limits the amount of direct tactile feedback received by a surgeon during a medical procedure. The ability to determine how much pressure to apply to a cutting or coagulation surface is paramount in procedures dealing with delicate and complicated anatomy. Electrosurgical instruments commonly used in these procedures may be undesirable due to the inability of the surgeon to accurately gauge the amount of pressure applied to a surface.

Presently, many disposable electrosurgical instruments are costly to manufacturers, hospitals, and patients, due to the number of parts present in these instruments. The need has arisen for disposable electrosurgical instruments that are cost effective, but yet give surgeons the control and range of motion necessary to successfully perform a wide variety of medical procedures.

The present invention overcomes the disadvantages of the prior art and provides the surgeon with a cost effective, yet efficiently flexible electrosurgical instrument.

### BRIEF SUMMARY OF THE INVENTION

One embodiment of the present invention comprises an electrosurgical medical device having a first finger cuff assembly comprising an elongated body having a distal end and a proximal end. An annular sleeve extends from the proximal end to the distal end and terminates at the distal end and provides for mounting on the distal portion of the surgeon's finger. An electrode is attached to, or is intrinsically part of, the first finger cuff assembly. This electrode is connected to at least one electrically conductive wire that is adapted to transmit electric current to the electrode.

A further embodiment of the present invention comprises an electrosurgical medical device with a second finger cuff assembly that houses a second electrode where the second finger cuff assembly cooperates opposably with the first finger cuff assembly to facilitate the transmission of an electric current between the two finger cuff assemblies.

A further embodiment of the present invention comprises an electrosurgical medical device comprising at least one finger cuff having an affixed grasping structure. The present invention comprises a means of using two finger cuffs in cooperation with one another where the affixed grasping structures are opposable to one another.

A still further embodiment of the present invention comprises an electrosurgical medical device where each of the extended surfaces of the grasping structure are extended distally in a finger forceps, flat form, nipper form, long form, short form, wide form, thin form, or looped form.

Another embodiment of the present invention comprises an electrosurgical medical device having insulative electrode pads that overlap the electrodes. The electrode pads are affixed to the electrodes and may function to focus or increase the size the amount of energy transmitted over a given area. One embodiment of the present invention features electrode pads affixed to the finger cuff with an adhesive tape. A plurality of electrode pads may be used to overlap the electrode until the desired transmittal aperture is achieved. A further embodiment of the present invention comprises an electrosurgical medical device having a plurality of electrodes located on each finger cuff assembly.

A further embodiment of the present invention comprises an electrosurgical medical device that utilizes either a bipolar or a monopolar current. One embodiment of the present invention comprises a monopolar current used in cooperation with a grounding system.

One embodiment of the present invention comprises an electrosurgical medical device having a full glove where the finger cuff assemblies are an intrinsic part of the full glove.

A still further embodiment of the present invention comprises an electrosurgical medical device having a delivery system that may be used to transfer irrigation fluid, pharmaceuticals, or other fluids to a desired area during a surgical procedure. The delivery system comprises at least one lumen and works in cooperation with the finger cuff assemblies. The delivery system also provides a lumen for transferring other medical instruments to the surgical site. Further, the delivery system may be used in cooperation with, or may function as, a viewing means to assist a physician during a surgical procedure. The viewing means may be a camera or a magnifying lens.

One embodiment of the present invention comprises an electrosurgical medical device having a scissor attachment located at the distal most portion of the finger cuff assemblies. Movement of the distal portion of the operator's fingers facilitates a scissors-like motion of the scissor attachment.

The present invention is useful for both open and laparoscopic surgeries, specifically hand-assisted laparoscopic surgery. In laparoscopic surgery the present invention is useful in combination with the methods as apparatus described in U.S. Pat. Nos. 6,110,154; 6,077,288 and 5,741,298, the contents of which are incorporated by reference herein. The present invention is also useful in combination with the LAP DISC® product marketed and sold by Ethicon Endo-Surgery, Inc., Cincinnati, Ohio.

### BRIEF DESCRIPTION OF THE FIGURES

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 is a perspective view of an electrosurgical medical device having two finger cuffs and electrodes in accordance with the present invention;

FIG. 2 is a perspective view of an alternate embodiment of an electrosurgical medical device having grasping finder cuffs in accordance with the present invention;

FIG. 3 is a perspective view of an alternate embodiment of an electrosurgical medical device in accordance with the present invention;

FIG. 4 is a perspective view of an alternate embodiment of an electrosurgical medical device in accordance with the present invention;

FIG. 5 is a magnified view of a plurality of electrodes attached to a first finger cuff assembly;

FIG. 6 is a perspective view of an electrosurgical medical device according to the present invention incorporating electrode pads;

FIG. 7 is a perspective view of an electrosurgical medical device according to the present invention incorporating a hinge;

FIG. 8 is a plan view of an electrosurgical medical device incorporating a fluid delivery and removal system;

FIG. 9 is a perspective view of an alternate embodiment of an electrosurgical medical device in accordance with the present invention;

FIG. 10 is a perspective view of an alternate embodiment of an electrosurgical medical device in accordance with the present invention;

FIG. 11 is a perspective view of an alternate embodiment of an electrosurgical medical device in accordance with the present invention;

FIG. 12 is a perspective view of an alternate embodiment of an electrosurgical medical device in accordance with the present invention;

FIG. 13 is a perspective view of an alternate embodiment of an electrosurgical medical device in accordance with the present invention; and

FIG. 14 is a perspective view of an alternate embodiment of an electrosurgical medical device in accordance with the present invention.

### DETAILED DESCRIPTION OF THE INVENTION

Before explaining the present invention in detail, it should be noted that the invention is not limited in its application or use to the details of construction and arrangement of parts illustrated in the accompanying drawings and description.

The illustrative embodiments of the invention may be implemented or incorporated in other embodiments, variations and modifications, and may be practiced or carried out in various ways. For example, the invention is described in conjunction with delivering RF electrical energy to a tissue site. Other energy sources, such as a laser, may be equally applicable to the disclosed invention.

Furthermore, unless otherwise indicated, the terms and expressions employed herein have been chosen for the purpose of describing the illustrative embodiments of the present invention for the convenience of the reader and are not for the purpose of limiting the invention.

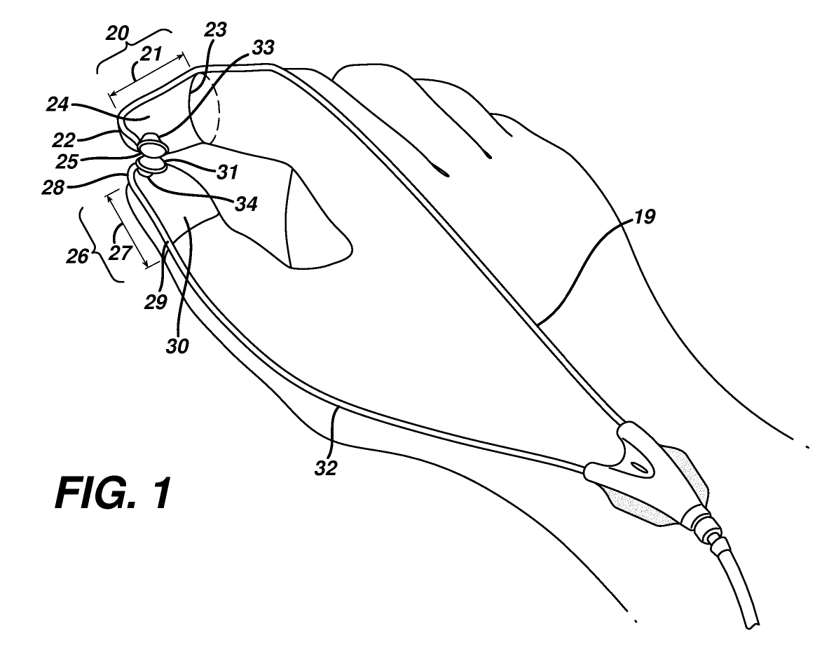

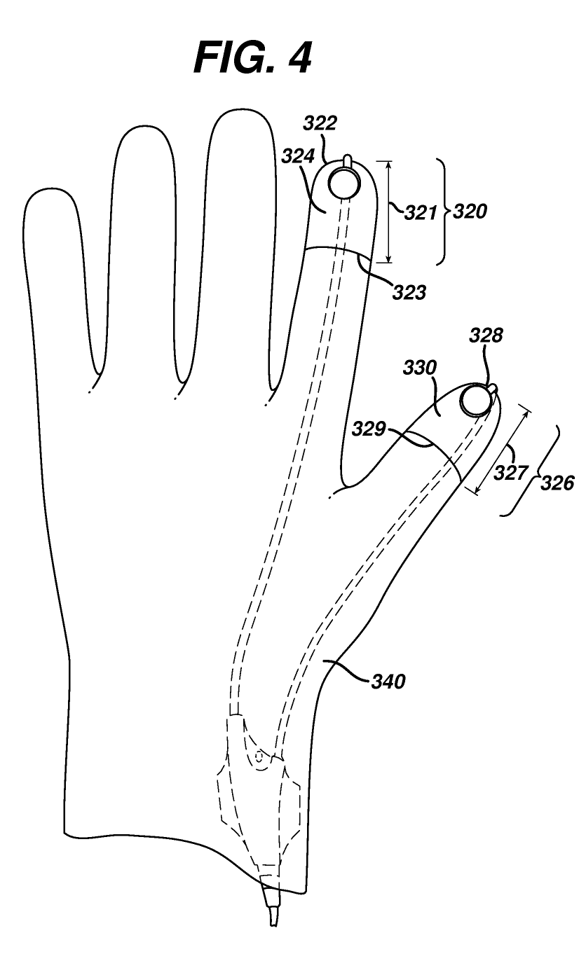

It is understood that any one or more of the following-described embodiments, expressions of embodiments, examples, methods, etc. can be combined with or are descriptive of any one or more of the other following-described embodiments, expressions of embodiments, examples, methods, etc. For example, and without limitation, any of the end effectors can be used in the embodiment disclosed in FIG. 4, and any of the end effectors may have the general characteristics of those described as relating to the embodiment of FIG. 1.

FIG. 1 illustrates one embodiment of the present invention comprising a first finger cuff assembly 20 having a first elongated body 21 that has a first distal end 22 and a first proximal end 23. A first annular sleeve 24 extends from the first proximal end 23 to the first distal end 22 terminating at the distal end 22. First annular sleeve 24 is adapted for mounting on the distal portion of the operator's finger. In one embodiment, the annular sleeve 24 is adapted to completely surround the distal portion of the surgeon's finger, though the invention contemplates a first annular sleeve 24 that partially surrounds the operator's finger or is part of a glove surrounding a portion, or all, of the operator's hand. First finger cuff assembly 20 may be sealed or unsealed at the distal most point of first finger cuff assembly, where the distal tip of the operator's finger may be fully exposed, partially exposed, or unexposed. First finger cuff assembly 20 may be constructed from plastic, metal, rubber, or other materials suitable for use in surgical procedures however, insulative material is preferable. First finger cuff assembly 20 further comprises a first electrode 25 that is affixed to the first finger cuff assembly 20. First electrode 25 may be attached in a removable fashion to first finger cuff assembly 20 by way of a temporary adhesive such as double-sided tape, or by other adhesive or mechanical (i.e. snap, clip, overmold, etc.) means known in the art. First electrode 25 may be permanently affixed to first finger cuff assembly 20, or may be an intrinsic feature of first finger cuff assembly 20. First electrode 25 may be constructed from stainless steel, aluminum, gold, platinum, silver, or other conductive materials suitable for use in surgical procedures. First electrode 25 may be coated with any suitable material commonly known in the art such as, for example, gold, platinum, silver, or partially coated with polytetrafluoroethylene (PTFE), Parylene, or ceramic to provide energy-focusing effects. A first electrically conductive wire 19 is connected to first electrode 25 and first electrically conductive wire 19 is adapted to transmit electric current to first electrode 20. First electrically conductive wire 19 may be constructed of any conductive material known in the art such as, for example, silver, copper, aluminum or stainless steel. The present invention may also comprise the use of a plurality of electrically conductive wires connected to first electrode 25 that are adapted to transmit electric current, as well as other means of transmitting electric current in the absence of electrically conductive wire. First electrode 25 may be adapted to transmit bipolar electrosurgical energy in cooperation with a second electrode 31,or alternatively, monopolar electrosurgical energy in cooperation with a grounding pad. In a monopolar embodiment of the present invention, first electrode 25 comprises at least one lead (not shown) via which it is connected to one pole of an RF generator (not shown). A ground, (not shown) used in cooperation with first electrode 25 adapted to transmit monopolar energy, may be affixed to second finger cuff assembly 26, may be a grounding pad attached to the patient's body, or may be any other suitable grounding means known in the art.

FIG. 1 further illustrates a second finger cuff assembly 26, which preferably has the same characteristics as the first finger cuff assembly 20, comprising a second elongated body 27 that has a second distal end 28 and a second proximal end 29. A second annular sleeve 30 extends from the first proximal end 29 to the first distal end 28 terminating at the distal end 28. Second annular sleeve 30 is also adapted for mounting on the distal portion of the operator's finger. A second electrically conductive wire 32 is connected to second electrode 31 and second electrically conductive wire 32 is adapted to transmit electric current to second electrode 31.

FIG. 1 further illustrates a first protective structure 33 and a second protective structure 34 that function to insulate the operator's fingers from possible damage caused by electrosurgical current. First protective structure 33 and second protective structure 34 may be constructed from any non-conductive material commonly known in the art such as, but not limited to, rubber, plastic, ceramic or foam.

First electrode 25 and second electrode 31 are opposable to one another and are adapted to be used in cooperation with one another to transmit electrosurgical energy. Alternating current between first electrode 25 and second electrode 31 functions to cut and/or coagulate, weld and/or ablate tissue located substantially between first electrode 25 and second electrode 31. The surgeon has the benefit of direct tactile feedback due to the close proximity of the electrodes to the fingers of the operator. Further, freedom of movement is limited only the surgeon's finger, hand and wrist range of motion. Cutting and/or coagulation, tissue welding and/or ablation varies depending on the electrosurgical energy transmitted through the first electrode 25 and the second electrode 31, as well as by the amount of opposable pressure applied by the finger tips of the operator. The present invention further comprises a sterile packaging environment. To provide the assurance of cleanliness to patients, the present invention comprises sterilizing the first finger assembly 20, second finger cuff assembly 26, first electrode 25, second electrode 31, and/or all other elements herein, where sterilization would be beneficial to the patient and/or operator. As would be evident to those skilled in the art and contemplated under this invention, additional finger cuff assemblies 20 may be placed on more than two fingers and electrically connected to alternate polarities. The user may then place three or more fingers on the tissue to be treated and manipulate the position of each finger to route therapeutic energy through the tissue as desired.

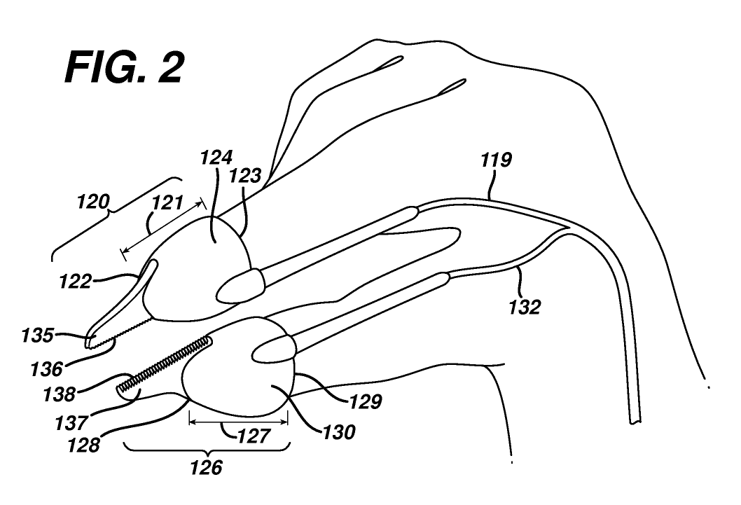

FIG. 2 illustrates one embodiment of the present invention comprising a first finger cuff assembly 120 having a first elongated body 121 that has a first distal end 122 and a first proximal end 123. A first annular sleeve 124 extends from the first proximal end 123 to the first distal end 122 terminating at the distal end 122. First annular sleeve 124 is adapted for mounting on the distal portion of the operator's finger. First finger cuff assembly 120 further comprises a first grasping structure 135 having a first extended surface 136. Surface 136 may be smooth, bumpy, serrated or any other type of surface finish as known in the medical device art, and as determined by the particular medical procedure. First grasping structure 135 may be attached in a removable fashion to first finger cuff assembly 120 by way of a temporary adhesive such as double-sided tape, or by other adhesive or mechanical (i.e. snap, clip, overmold, etc.) means known in the art. First grasping structure 135 may be permanently affixed to first finger cuff assembly 120, removable from first finger cuff assembly 120, or may be an intrinsic feature of first finger cuff assembly 120. One embodiment of the present invention comprises a removable first grasping structure 135 that may be interchanged with any other grasping structure or suitable end effector. First grasping structure 135 may be removably fastened to first finger cuff assembly 120 by a key snap with a spring release, an open ended T-slot, a screw-on attachment, or by other suitable attachment means. The present invention further contemplates a permanent attachment of first grasping structure 135 to first finger cuff assembly 120. The means for permanently affixing first grasping structure 135 to first finger cuff assembly 120 include a snap on fit, a locking screw, a permanent adhesive, or other means of achieving a permanent attachment known in the art. First grasping structure 135 may be constructed from stainless steel, gold, platinum, silver aluminum, titanium, plastic, ceramic or other materials suitable for use in surgical procedures. First extended surface 136 may be constructed from stainless steel, gold, silver, platinum, aluminum, or other conductive materials suitable for use in surgical procedures. Further, first extended surface 136 or grasping structure 135 may be made of a malleable conductive material to allow the user to bend or shape the surface 136 or structure 135 to conform to the tissue profile encountered. Bending or shaping may be performed by means of hand tools or by hand.

Grasping structures may also be used for electrosurgical applications. First extended surface 136 may be coated with any suitable material commonly known in the art such as, for example, gold, silver, PTFE, Parylene, or ceramic. A first electrically conductive wire 119 is connected to first extended surface 136 and first electrically conductive wire 119 is adapted to transmit electric current to first extended surface 136. First electrically conductive wire 119 may be constructed of any conductive material known in the art such as, for example, stainless steel or silver (note that these materials are not required to be conductive in this embodiment. If the finger cuffs are simply graspers with no RF activation, they can be made of non-conductive materials). The present invention comprises the use of a plurality of electrically conductive wires connected to first extended surface 136 that are adapted to transmit electric current, as well as other means of transmitting electric current in the absence of electrically conductive wire (i.e. flexcircuit, conductive polymers, conductive films, etc.)(magnetic flux generator).

First extended surface 136 may be adapted to transmit bipolar electrosurgical energy or monopolar electrosurgical energy.

FIG. 2 further illustrates a second finger cuff assembly 126 having a second elongated body 127 that has a second distal end 128 and a second proximal end 129. A second annular sleeve 130 extends from the second proximal end 129 to the second distal end 128 terminating at the second distal end 128 and comprising a second grasping structure 137. A second electrically conductive wire 132 is connected to second extended surface 138 and second electrically conductive wire 132 is adapted to transmit electric current to second extended surface 138.

First extended surface 136 and second extended surface 138 are opposable to one another and are adapted to be used in cooperation with one another to transmit electrosurgical energy. Alternating current between first extended surface 136 and second extended surface 138, depending on instrument configuration and generator settings, functions to cut and/or coagulate, weld and/or ablate tissue located substantially between first extended surface 136 and second extended surface 138. The operator of the present invention has the benefit of direct tactile feedback due to the close proximity of the electrodes to the fingers of the operator. Cutting and/or coagulation, welding and/or ablation varies depending on the electrosurgical energy transmitted through the first extended surface 136 and the second extended surface 138, as well as by the amount of opposable pressure applied by the finger tips of the operator and instrument geometry. First extended surface 136 and second extended surface 138 are preferably 0.1-4 mm wide and 1-100 mm in length, although other widths and lengths desirable for use in a surgical application are consistent with the present invention.

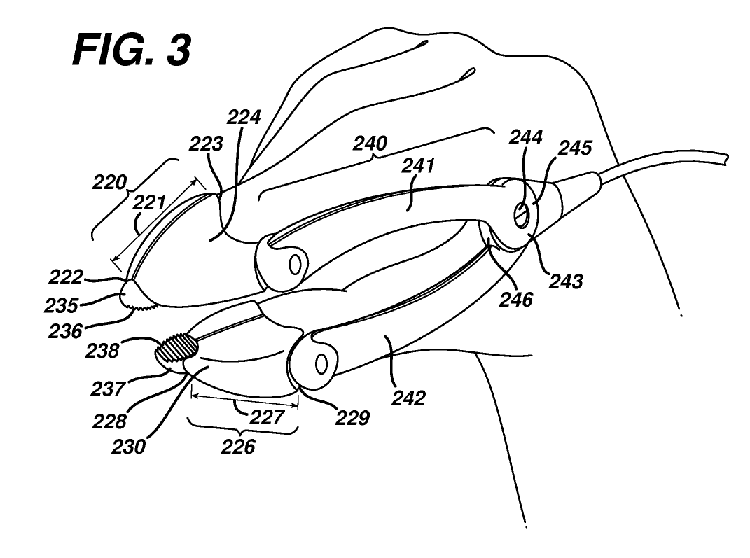

FIG. 3 illustrates an alternate embodiment of the present invention, known as a nipper, comprising a first and second finger cuff assembly 220, 226. Assembly 220 comprises a first elongated body 221 that has a first distal end 222 and a first proximal end 223. A first annular sleeve 224 extends from the first proximal end 223 to the first distal end 222 terminating at the distal end 222. First annular sleeve 224 is adapted for mounting on the distal portion of the surgeon's finger. In one embodiment, the annular sleeve 224 is adapted to completely surround the distal portion of the operator's finger, though the present invention contemplates first annular sleeve 224 that partially surrounds the operator's finger or is part of a glove surrounding a portion, or all, of the operator's hand. First finger cuff assembly 220 may be constructed from plastic, metal, rubber, or other materials suitable for use in surgical procedures however, preferably an insulative material. First finger cuff assembly 220 further comprises a first grasping structure 235 having a first extended surface 236. A first electrically conductive wire (not shown) may be connected to the extended surface 236 and adapted to transmit RF energy.

A second finger cuff assembly 226 comprises a second elongated body 227 that has a second distal end 228 and a second proximal end 229. A second annular sleeve 230 extends from the second proximal end 229 to the second distal end 228 terminating at the second distal end 228. Second annular sleeve 230 is adapted for mounting on the distal portion of the operator's finger. A second electrically conductive wire (not shown) is connected to second extended surface 238 and second electrically conductive wire (not shown) is adapted to transmit electric current to second extended surface 238.

FIG. 3 further illustrates an integral assembly system 240 having a first connecting member 241, a second connecting member 242 and a connection joint 243. Integral system 240 is helpful to maintain cuff assemblies 220 and 226 in a specific relationship for optimal operation. The distal most portion of first connecting member 241 is affixed to the first finger cuff assembly 224, and the proximal portion of the first connecting member 241 is affixed to connection joint 243. The distal most portion of the second connecting member 242 is affixed to the second finger cuff assembly 230, and the proximal portion of the second connecting member 242 is affixed to the connection joint 224. Preferably, first connecting member 241 and second connecting member 242 house electrically conductive wires (not shown) Connecting members 241, 242 may be affixed to finger cuff assemblies by a weld, a hinge, a snap fit, or other means of attachment known in the art. The proximal portions of connecting members 241, 242 may be affixed to connection joint 243 by a weld, a hinge, a snap fit, or other means of attachment known in the art. In one embodiment of the present invention, connection joint 243 is a hinge having a central pin 244. Connection joint 243 comprises a first rotating member 245 and a second rotating member 246 providing a means for rotating first rotating member 245 and second rotating member 246 in relating to one another around central pin 244. Rotational movement of first rotating member 245 and second rotating member 246 in relation to one another, around central pin 244 allows first finger cuff 224 and second finger cuff 230 to move opposably.

FIG. 4 illustrates a further embodiment of FIG. 1 in which first finger cuff assembly 320 and second finger cuff assembly 326 are integral with a glove 340. Glove 340 may be constructed from rubber, plastic, or other insulative material suitable for use in a surgical procedure. Glove 340 may fully cover the entire hand of the operator, or may be designed to partially cover the hand of the operator. First finger cuff assembly 320 has an elongated body 321 that has a distal end 322 and a proximal end 323. An annular sleeve 324 extends from the proximal end 323 to the distal end 322 terminating at the distal end 322. Annular sleeve 324 is adapted for mounting on the distal portion of the operator's finger via glove 340. Second finger cuff assembly 326 has a second elongated body that has a first distal end 328 and a first proximal end 329. A second annular sleeve 330 extends from the first proximal end 329 to the first distal end 328 terminating at the distal end 328.

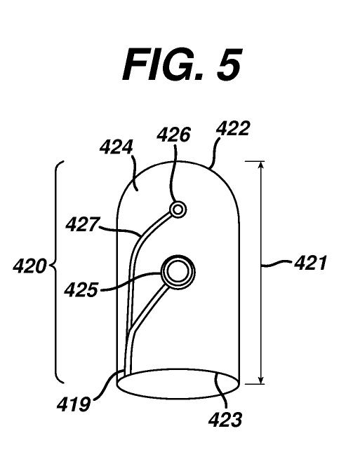

FIG. 5 illustrates an exemplary embodiment of a finger cuff and electrode assembly. Finger cuff assembly 420 comprises an elongated body 421 that has a distal end 422 and a proximal end 423. An annular sleeve 424 extends from the proximal end 423 to the distal end 422 terminating at the distal end 422. Annular sleeve 424 is adapted for mounting on the distal portion of the operator's finger. Finger cuff assembly 420 further comprises a first electrode 425 that may be attached in a removable fashion to finger cuff assembly 420 by way of a temporary adhesive such as double-sided tape, or by other adhesive or mechanical means (i.e. snap, clip, overmold, etc.) means known in the art. Electrode 425 may be permanently affixed to finger cuff assembly 420, or may be an intrinsic feature of finger cuff assembly 420. Electrode 425 may be constructed from stainless steel, gold silver, aluminum, platinum, or other conductive materials suitable for use in surgical procedures. Electrode 425 may be coated with any suitable material commonly known in the art such as, for example, gold, silver, PTFE, Parylene, or ceramic. An electrically conductive wire 419 is connected to electrode 425 and electrically conductive wire 419 is adapted to transmit electric current to first electrode 420.

FIG. 5 illustrates a second electrode 426 that is affixed to the first finger cuff assembly 420. The present invention further contemplates the use of a plurality of electrodes in cooperation with finger cuff assembly 420. The present invention comprises the use of a plurality of electrodes of varying diameter, surface area, and shape in cooperation with first finger cuff assembly 420. The present invention comprises electrodes 425 and 426 with shapes such as, but not limited to, circular, oval, square, rectangle, triangular, asymmetrical, symmetrical, or other shapes suitable for use in a surgical procedure.

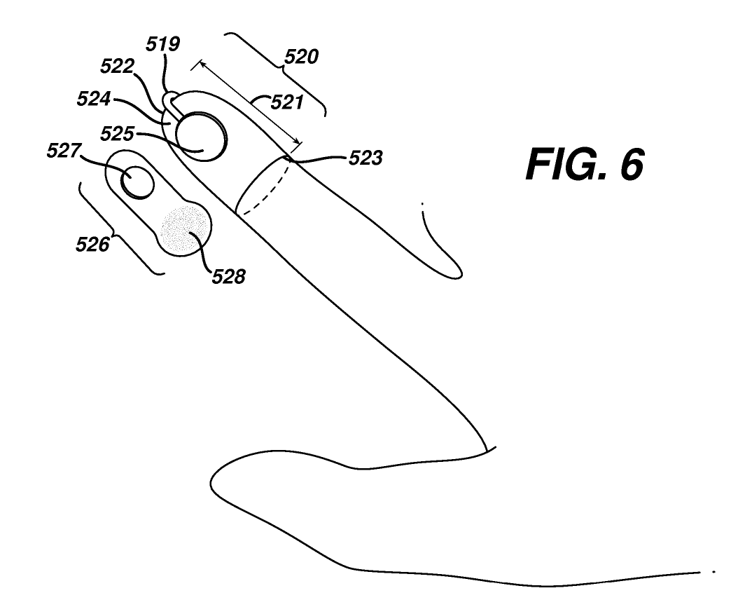

FIG. 6 illustrates one embodiment of the present invention a first finger cuff assembly 520 having a first elongated body 521 that has a first distal end 522 and a first proximal end 523. A first annular sleeve 524 extends from the first proximal end 523 to the first distal end 522 terminating at the distal end 522. First annular sleeve 524 is adapted for mounting on the distal portion of the operator's finger. In one embodiment, the annular sleeve 524 is adapted to completely surround the distal portion of the operator's finger, though the present invention comprises a first annular sleeve 524 that partially surrounds the operator's finger or is part of a glove surrounding a portion of the operator's hand. First finger cuff assembly 520 may be constructed from plastic, metal, rubber, or other materials suitable for use in surgical procedures however, preferably an insulative material. First finger cuff assembly 520 further comprises a first electrode 525 that is affixed to the first finger cuff assembly 520. First electrode 525 may be attached in a removable fashion to first finger cuff assembly 520 by way of a temporary adhesive such as double-sided tape, or by other adhesive or mechanical (i.e. snap, clip, overmold, etc.) means known in the art. First electrode 525 may be permanently affixed to first finger cuff assembly 520, or may be an intrinsic feature of first finger cuff assembly 520. First electrode 525 may be constructed from stainless steel, gold, silver, platinum, aluminum, or other conductive materials suitable for use in surgical procedures. First electrode 525 may be coated with any suitable material commonly known in the art such as, for example, gold, silver, PTFE, Parylene, or ceramic. A first electrically conductive wire 519 is connected to first electrode 525 and first electrically conductive wire 519 is adapted to transmit electric current to first electrode 525. First electrically conductive wire 519 may be constructed of any conductive material known in the art such as, for example, copper. The present invention comprises the use of a plurality of electrically conductive wires connected to first electrode 525 that are adapted to transmit electric current, as well as other means of transmitting electric current in the absence of electrically conductive wire. First electrode 525 may be adapted to transmit bipolar electrosurgical energy or monopolar electrosurgical energy.

FIG. 6 further illustrates an electrode pad 526 used in cooperation with first finger cuff assembly 520. The present invention comprises a means of affixing electrode pad 526 to first finger cuff assembly 520 by way of double stick tape, glue, or by other suitable adhesives known in the art. Electrode pad 526 comprises at least one secondary electrode 527 that is adapted to transmit electric current. When electrode pad 526 is affixed to first finger cuff assembly 520, first electrode 525 and secondary electrode 527 are substantially aligned so as to transfer electrosurgical energy from first electrode 525 through secondary electrode 527. Electrode pad 526 may have an adhesive region 528 that is affixed to first finger cuff assembly 520 is such a manner that secondary electrode 527 of electrode pad 526 imbricates first electrode 525 of first finger cuff assembly 520. Electrode pad 526 may be constructed of any material suitable for use in surgical procedures such as, for example, rubber or plastic however, preferably an insulative material. The present invention further comprises the use of a plurality of electrode pads 526 in cooperation with one another. Electrode pad 526 may comprises a plurality of secondary electrodes.

The electrodes 527 may be stacked on top of each other and separated by an peelable insulative layer to provide multiple peel-off sections of the same area so that when on electrode becomes clogged with burned tissue, the surgeons peels that pad off to expose a new one. Alternatively, electrode 527 may be covered by a number of peelable insulating members so that as the surgeon peels away each insulting layer, a larger portion of the base electrode is exposed to the tissue.

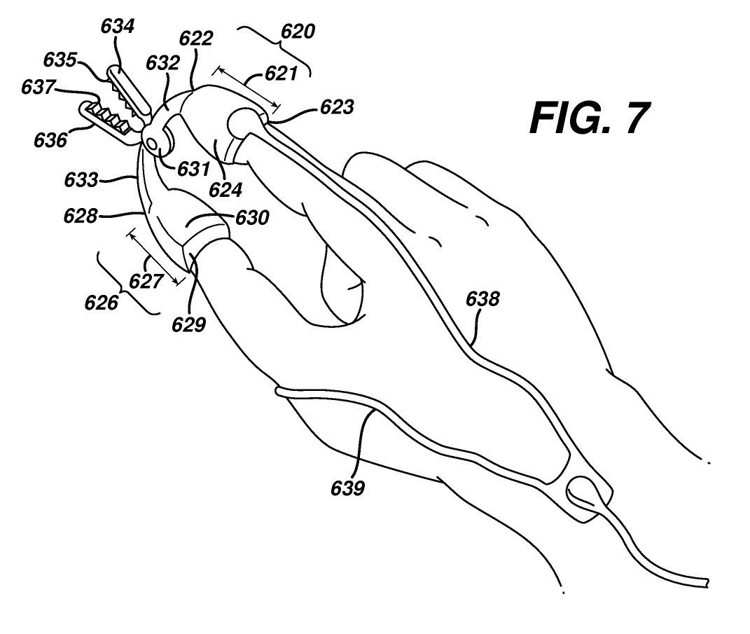

FIG. 7 illustrates another embodiment of the present invention comprising a first finger cuff assembly 620 having a first elongated body 621 that has a first distal end 622 and a first proximal end 623. A first annular sleeve 624 extends from the first proximal end 623 to the first distal end 622 terminating at the distal end 622. First annular sleeve 624 is adapted for mounting on the distal portion of the operator's finger.

FIG. 7 illustrates a second finger cuff assembly 626 having a second elongated body 627 that has a second distal end 628 and a second proximal end 629. A second annular sleeve 630 extends from the first proximal end 629 to the first distal end 628 terminating at the distal end 628. Second annular sleeve 630 is adapted for mounting on the distal portion of the operator's finger.

FIG. 7 further discloses a hinge 631 located distally in relation to the distal most portion of the operator's fingers. The present invention comprises a means of connecting hinge 631 to first finger cuff assembly 620 by way of a first connection structure 632. The present invention comprises a means of connecting hinge 631 to second finger cuff assembly 626 by way of a second connection structure 633. The proximal most portion of first connection structure 632 is affixed to the hinge 631, and the distal most portion of first connection structure 632 is affixed to first finger cuff assembly 620. The proximal most portion of second connection structure 633 is affixed to hinge 631, and the distal most portion of first connection structure 632 is affixed to second finger cuff assembly 626. The present invention comprises a first effector feature 634, having a first extended surface 635, located distally to hinge 631 and a second effector feature 636 having a second extended surface 637, located distally to hinge 631. First effector feature 634 comprises a first effector electrode (not shown) housed within first effector feature 634. Second effector feature 636 comprises a second effector electrode (not shown) housed within second effector feature 636. The present invention comprises a first at least one electrically conductive wire 638 adapted to transmit electric current to the first effector electrode (not shown) and a second at least one electrically conductive wire 639 adapted to transmit electric current to the second effector electrode (not shown). The present invention comprises a means of moving opposably first effector feature 634 and second effector feature 636 by opposably moving the operator's fingers, in cooperation with hinge 631, to facilitate a scissors-like opposable motion. Hinge 631 may be a lever, a spring, or other means of providing suitable opposable motion.

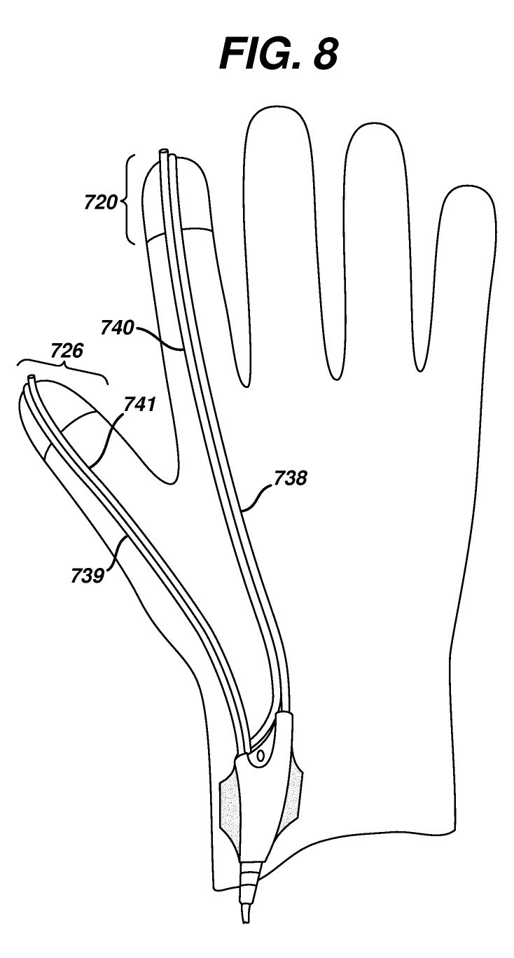

FIG. 8 illustrates still another embodiment of the present invention comprising a first finger cuff assembly 720 and a second finger cuff assembly 726 including a first at least one electrically conductive wire 738 and a second at least one electrically conductive wire 739 used in cooperation with a first lumen 740 and a second lumen 741. The present invention comprises the use of first lumen 740 independently, in cooperation with second lumen 741, or in cooperation with a plurality of other lumen. Lumens 740 and 741 may be designed for irrigation, suction, pharmacological delivery, as a means to deliver a viewing device, as a means to deliver a snare, or as a means to deliver any suitable medical device to the area of operation. Alternatively, lumens 740 and 741 may provide conduits for optical fibers to provide for improved illumination at the user's fingertips. The illumination may also provide means for a status indicator; for example, the presence, absence or modulation of color or intensity of the illumination would provide feedback to the user regarding energy level applied to the tissue, tissue impedance or other parameters. Lumens 740 and 741 may be extendable, retractable, or permanently affixed at a desired location. Lumens 740 and 741 may be designed to perform the same function such as, for example, irrigation, or lumens 740 and 741 may be designed to perform different function such as, for example, suction and irrigation, respectively. Lumens 740 and 741 may be located on a single finger cuff, or may be located on opposable first and second finger cuffs 720 and 726 as illustrated in FIG. 8. Lumens 740 and 741 may be constructed from plastic, rubber, metal, or other material suitable for use in a surgical procedure, however preferably an insulative material. Irrigation comprises the delivery of water, dyes, radioisotopes, or other fluids beneficial for use in a surgical procedure.

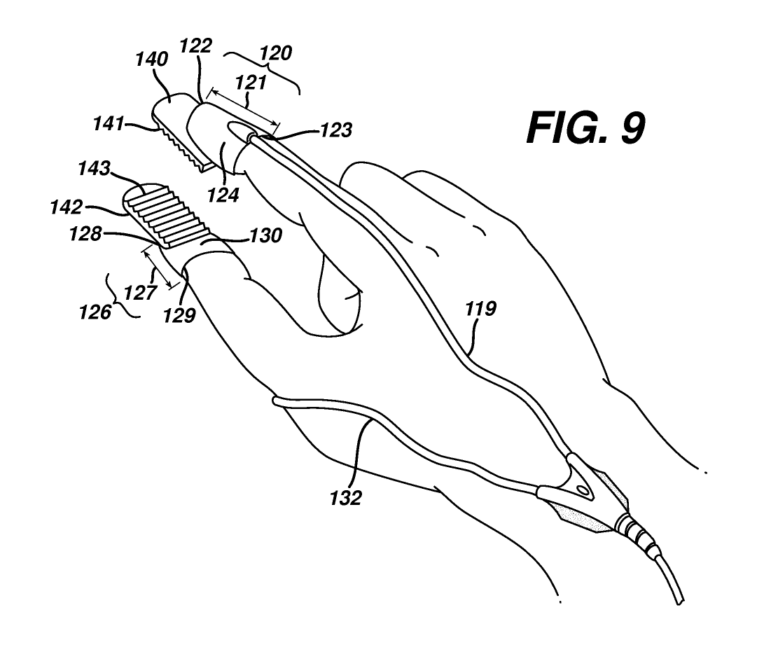

FIGS. 9-13 illustrate first and second finger cuff assembles 120 and 126 of FIG. 2 and alternate embodiments of the grasping and cutting and coagulating structure of FIG. 2. FIG. 9 discloses finger cuff assemblies 120 and 126 having wide grasping structures 140, 142 with wide extended surfaces 141 and 143. Wide extended surfaces 141 and 142 are preferably 4-20 mm wide and 1-60 mm in length, however other widths and lengths preferable for use in a surgical procedure are consistent with the present invention. The present invention further comprises the removal or deactivation of material substantially in the center of first wide extended surface 141 and second wide extended surface 143 in order to facilitate a looped extended surface (not shown).

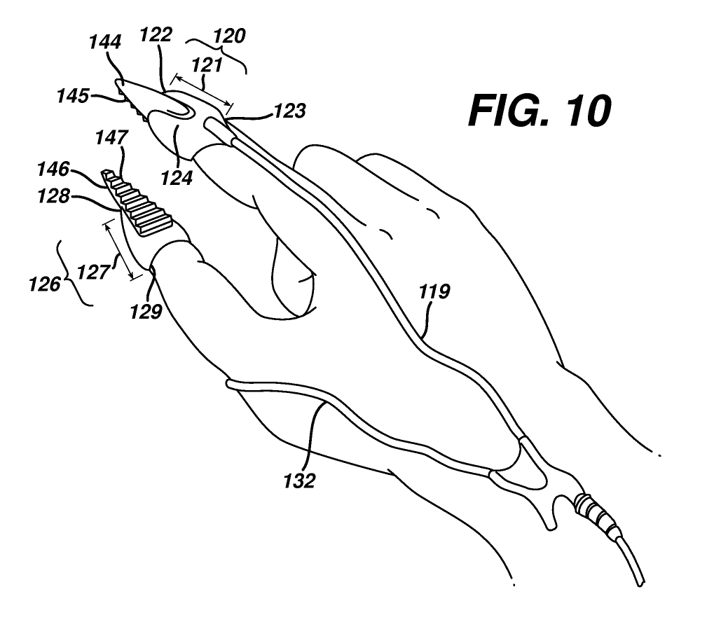

FIG. 10 discloses finger cuff assemblies 120 and 126 having short grasping structures 144, 146 with corresponding short extended surfaces 145,147. Short extended surfaces 145, 147 are preferably 1-20 mm wide and 1-10 mm in length, however other widths and lengths preferable for use in a surgical procedure are consistent with the present invention and may be constructed in a variety of forms such as, for example, tapered or curved.

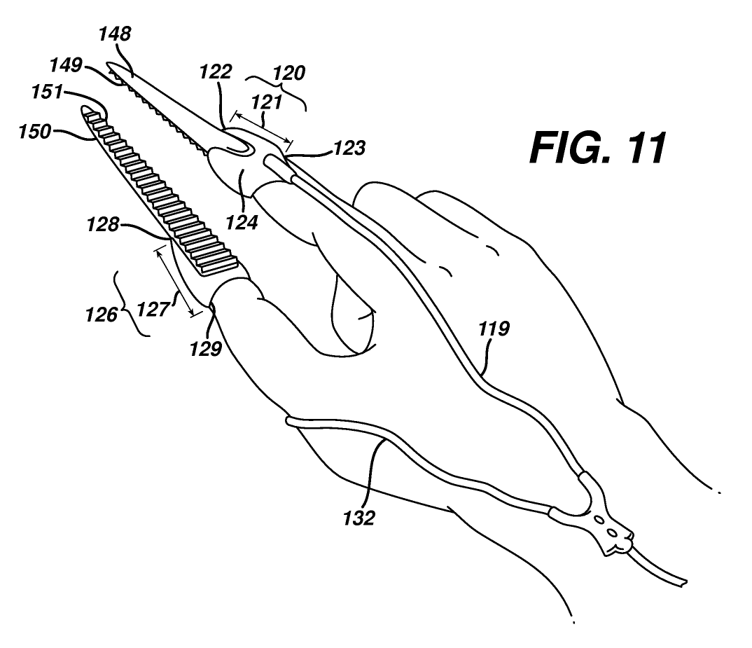

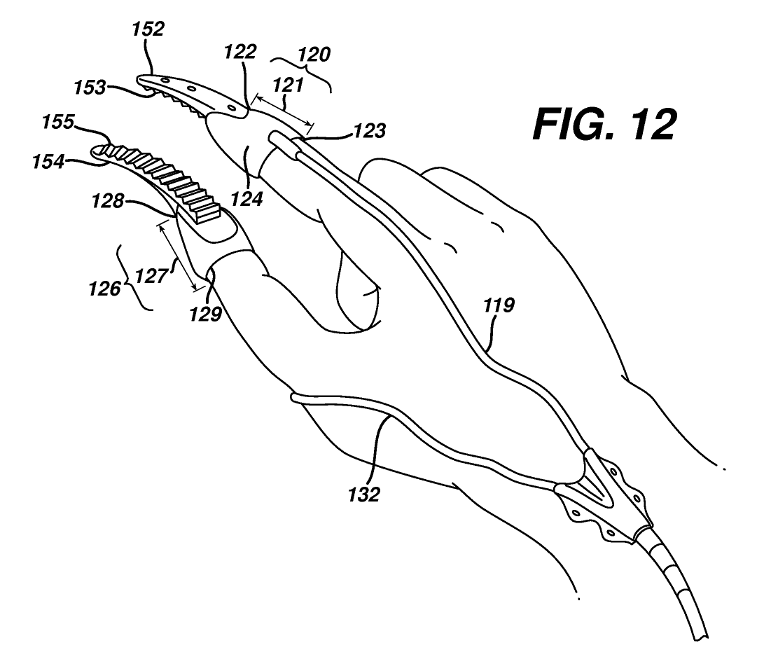

FIG. 11 illustrates finger cuff assemblies 120 and 126 having narrow grasping structures 148, 150 with extended surfaces 149 and 151. Extended surfaces 149 and 151 are preferably 1-20 mm wide and 11-100 mm in length, however other widths and lengths preferable for use in a surgical procedure are consistent with the present invention. First long extended surface 149 and second long extended surface 151 may be constructed in a variety of forms such as, for example, tapered or curved as disclosed in FIG. 12.

In FIG. 12, curved extended surfaces 153 and 155 are preferably 1-20 mm wide and 1-100 mm in length, however other widths and lengths preferable for use in a surgical procedure are consistent with the present invention.

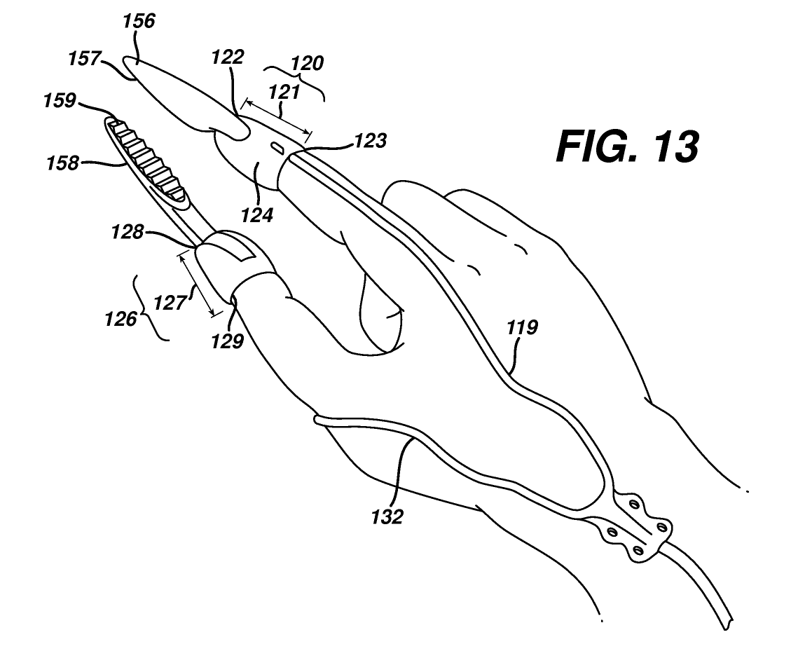

FIG. 13 discloses finger forceps 156, 158 having extended surfaces 157, 159 preferably 1-20 mm wide and 1-100 mm in length, however other widths and lengths preferable for use in a surgical procedure are consistent with the present invention.

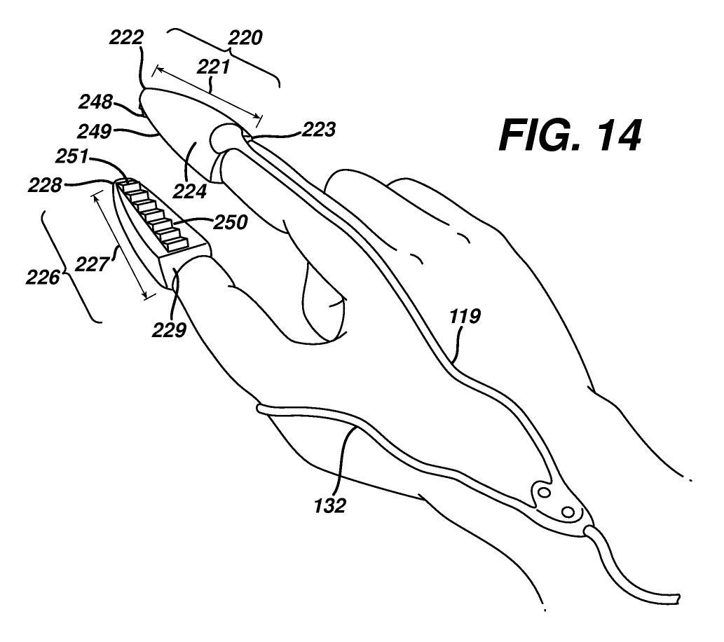

FIG. 14 illustrates first and second finger cuff assembles 220 and 226 of FIG. 3 and alternate embodiments of the grasping and cutting and coagulating structure of FIG. 3 designated FIG. 14 illustrates distal ends 222 and 228 having extended surfaces 249, 251, respectively.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. For example, the present invention may be adapted for use with ultrasound, cryonics, laser, mechanical devices such as clip appliers, scissors, or with other means of performing surgical procedures known in the art. The present invention may be used in cooperation with other medical devices such as, but not limited to, a snare, a sensor, a thermal sensor, a plurality of sensors, or an endoscope. Moreover, the structure of each element associated with the present invention can be alternatively described as a means for providing the function performed by the element. Accordingly, it is intended that the invention be limited only by the spirit and scope of the appended claims.

We claim:

1. A medical device comprising:
(a) a first finger cuff assembly for mounting on the distal portion of an operator's finger;
(b) a first electrode attached to the first finger cuff assembly; and
(c) an electrically conductive wire connected to the electrode and a source of electrosurgical energy.

2. The medical device of claim 1, wherein the first electrode is used in conjunction with an electrosurgical grounding pad.

3. The medical device of claim 2, wherein the electric current is monopolar electrosurgical energy.

4. The medical device of claim 1, further comprising:
(a) a second finger cuff assembly for mounting on the distal portion of the operator's finger;
(c) a second electrode attached to the second finger cuff assembly; and
(d) a second electrically conductive wire connected to the second electrode and source of electrosurgical energy.

5. The medical device of claim 4, wherein the finger cuff assembly is opposable to the second finger cuff assembly.

6. The medical device of claim 5, wherein the first electrode is opposable to the second electrode.

7. The medical device of claim 6, wherein electric current is transmitted between the first electrode and the second electrode.

8. The medical device of claim 7, wherein the electric current is bipolar electrosurgical energy.

9. The medical device of claim 4, wherein the first finger cuff and second finger cuff further comprise a first grasping structure and a second grasping structure, respectively.

10. The medical device of claim 9, wherein the first grasping structure and the second grasping structure are removably attached to the first and second finger cuffs.

11. The medical device of claim 9, wherein the first grasping structure is opposable to the second grasping structure.

12. The medical device of claim 11, wherein electric current is transmitted between the first grasping structure and the second grasping structure.

13. The medical device of claim 4, further comprising an integral assembly system comprising a first connecting member, a second connecting member, and a connection joint, wherein the first connecting member is affixed to the first finger cuff assembly and the connection joint; and the second connecting member is affixed to the second finger cuff assembly and the connection joint.

14. The medical device of claim 13, wherein the connection joint is a hinge.

15. The medical device of claim 13, wherein the first finger cuff assembly is opposable to the second finger cuff assembly.

16. The medical device of claim 13, wherein the finger cuffs accept monopolar scissor jaws as a tissue effector.

17. The medical device of claim 1, further comprising a first electrode pad, wherein the first electrode pad is affixed to the first electrode, wherein the first electrode pad comprises at least one secondary electrode adapted to transmit electric current.

18. The medical device of claim 17, wherein the first electrode pad is affixed to the first electrode with an adhesive.

19. The medical device of claim 17, wherein the at least one secondary electrode has a substantially smaller surface area than the first electrode.

20. The medical device of claim 1, wherein the first finger cuff further comprises I lumen for accepting a viewing means.

21. The medical device of claim 20, wherein the viewing means is a camera.

22. The medical device of claim 1, wherein the first finger cuff further comprises a lumen for accepting a suction means.

23. The medical device of claim 1, wherein the first finger cuff further comprises a lumen for accepting an irrigation means.

24. The medical device of claim 1, further comprising a pharmacological delivery lumen.

24. The medical device of claim 1, further comprising a glove, wherein the first finger cuff assembly is integral with the glove.

25. The medical device of claim 9, wherein the first grasping structure is malleable.

26. A medical device comprising:
(a) a first finger cuff assembly for mounting on the distal portion of an operator's finger and a second finger cuff assembly for mounting on the distal portion of an operator's finger;
(b) a first grasping device attached to the first finger cuff assembly; and
(c) a second grasping device attached to the second finger cuff assembly.

27. The medical device of claim 26 wherein the first grasping device comprises at least one electrode adapted to transmit electric current.

28. The medical device of claim 26 wherein the first and second grasping devices each comprise at least one electrode adapted to transmit electric current.

29. The medical device of claim 1 wherein the first finger cuff further comprises a lumen for accepting a laser fiber.

30. The medical device of claim 1, further comprising a first electrode pad, wherein the first electrode pad is affixed to the first electrode, wherein the first electrode pad comprises at least one removable insulating layer that extends at least over the first electrode.

31. The medical instrument of claim 30 wherein said electrode pad has a plurality of removable insulating layers, the removal of each allows a larger area of said first electrode to be exposed to the patient.

32. The medical device of claim 1, further comprising a plurality of electrode pads, wherein the first electrode pad is affixed to the first electrode, and wherein the first electrode pad comprises at least one secondary electrode adapted to transmit electric current.

33. The medical device of claim 32 wherein each of the plurality of removable electrode pads is affixed to the electrode beneath it and wherein each electrode pad comprises at least one secondary electrode adapted to transmit electric current.

34. The medical device of claim 33 wherein each of the plurality of removable electrode pads can be removed by the user to expose the pad directly beneath.
